(19) United States
(12) Patent Application Publication
(10) Pub. No.: US 2006/0141031 A1
(43) Pub. Date: Jun. 29, 2006

(54) Orally disintegrating pharmaceutical compositions with sensory cue agents

(76) Inventors: Dennis G. Nelson, Mountain Lakes, NJ (US); William Michael Nichols, Somerset, NJ (US)

Correspondence Address: Barry H. Jacobsen;Legal Division, Warner-Lambert Company, LLC, 201 Tabor Road, Morris Plains, NJ, 07950, US

(21) Appl. No.: 11/303,822

(22) Filed: Dec. 16, 2005

Related U.S. Application Data
(60) Provisional application No. 60/638,526, filed on Dec. 23, 2004.

(51) Int. Cl. A61K 9/20

(52) U.S. Cl. 424/464

(57) ABSTRACT

The present invention relates to pharmaceutical compositions that disintegrate in the mouth and include at least one systemically active pharmaceutical ingredient in a therapeutically effective amount and at least one sensory cue agent in a sensory effective amount.

This application claims priority to U.S. Provisional Application 60/638,526, filed Dec. 23, 2004.

### FIELD OF THE INVENTION

The present invention relates to pharmaceutical compositions having a sensory cue agent and methods of using such compositions. More particularly, the invention relates to an orally fast disintegrating pharmaceutical composition having at least one systemically active pharmaceutical ingredient in a therapeutically effective amount and a sensory cue agent in a sensory imparting effective amount.

### BACKGROUND OF THE INVENTION

Pharmaceutical compositions that disintegrate in the mouth are beneficial for many reasons. Their characteristic advantages such as administration without liquid, anywhere, anytime lead to their suitability in clinical situations where patients have difficulty swallowing, such as children, the elderly, those with neurological disorders, the mentally ill, the bed-ridden, patients without teeth, and patients who do not have easy access to liquids. They are also suitable in patients whose liquid intake is restricted, such as prior to bedtime in elderly patients, patients in whom nocturia is problematic, or prior to surgery. Additionally, the inconvenience, lack of discreteness, and effect on patient compliance caused by the need to take tablets with liquid is problematic where water is unavailable or where the patient is unable to swallow because of a sore throat or an allergic attack.

Orally disintegrating pharmaceutical compositions typically include a systemically active pharmaceutical ingredient (API). Systemic agents typically take thirty or more minutes to affect the patient since the pharmacological effect from the API is not achieved until the API is released systemically, thereby resulting in a delayed onset of action.

Sensory agents are known for use in liquid pharmaceutical compositions and confectionary type solids such as lozenges or gums. However, for pharmaceutical compositions formulated to disintegrate quickly in oral cavity (i.e., buccal or sublingual), the combination of sensory agents and systemic APIs has not been done.

Thus, it would be desirable to have a fast disintegrating pharmaceutical composition with a systemically API that provides an immediately perceivable therapeutic effect. Accordingly, there is a need for a pharmaceutical composition that disintegrates in the buccal cavity and provides a sensory cue agent in a sensory effective amount to impart an immediately perceivable relief and provide a systemic API in a therapeutically effective amount.

### SUMMARY OF THE INVENTION

Various embodiments of the present invention provide for pharmaceutical compositions including at least one systemically active pharmaceutical ingredient in a therapeutically effective amount; and at least one sensory cue agent in a sensory cue effective amount. The pharmaceutical compositions may be designed to disintegrate in the buccal cavity in less than about 60 seconds.

Other embodiments of the present invention provide for pharmaceutical compositions including at least one systemically active pharmaceutical ingredient in a therapeutically effective amount. The active pharmaceutical ingredient may be selected from the group consisting of phenylephrine, pseudoephedrine, dextromethorphan, diphenhydramine and combinations thereof. The composition may include at least one sensory cue agent in a sensory cue effective amount. The sensory cue agents may be selected from the group consisting of menthol oil, menthol crystals, mannitol, eucalyptus oil, eucalyptol, thymol, camphor, spearmint, cinnamon, mint, ginger, wintergreen (methyl salicylate), peppermint, carboxamides, acyclic carboxamides, 3-1-menthoxy propane-1,2-diol, N-substituted-p-menthane-3-carboxamides, N-ethyl-p-menthane-3-carboxamide, N,2,3-trimethyl-2-isopropylbutanamide and combinations thereof. The pharmaceutical composition may be a tablet designed to disintegrate in the buccal cavity in less than about 60 seconds.

Further embodiments provide methods for treating ailments, such as upper respiratory indications, in humans and animals, including the step of administering such compositions to patients, inlcuding patients in need of such treatment.

Still further embodiments provide for packaged kits for a patient including a housing of a plurality of oral dosage forms including a pharmaceutical composition including at least one systemically active pharmaceutical ingredient in a therapeutically effective amount; and at least one sensory cue agent in a sensory cue effective amount. The pharmaceutical composition may be designed to disintegrate in the buccal cavity in less than about 60 seconds, and may include instructions for carrying out drug administration therewith.

### DETAILED DESCRIPTION OF THE INVENTION

It has surprisingly been found that the administration of at least one sensory cue agent together with at least one systemic API in a therapeutically effective amount results in a two-pronged, synergistic therapeutic effect. A sensory cue agent provides a sensory cue to the mouth, throat, nasal and/or sinus passages so that the pharmaceutical composition may be perceived by the patient as immediately acting to alleviate an ailment. Additionally, a sensory cue agent improves mouthfeel perception, is organoleptically pleasing, and thereby improves patient compliance. Thus, a patient is provided with a sensation of immediate relief as well as a systemic API that systemically alleviates the conditions causing the ailment.

Various embodiments of the present invention provide a pharmaceutical composition, such as an orally disintegrating dosage form, that includes at least one systemic API in a therapeutically effective amount and at least one sensory cue agent in a sensory imparting effective amount. A useful orally disintegrating dosage form includes a fast disintegrating dosage form. Several embodiments of the present invention provide a pharmaceutical composition such as a fast disintegrating dosage form (hereinafter ‘FDDF’) which disintegrates quickly in the mouth/buccal cavity. The term fast disintegrating dosage form as used herein means that disintegration is to be considered as greater than about 95% of the dosage form disintegrated in water at 37° C. after about 3 minutes. In certain embodiments, about 90% of the dosage form has been disintegrated after about 1 minute. The disintegration time of a FDDF can be determined by placing a tablet into a mouth and measuring the time period taken for complete disintegration of the tablet by saliva with no additional liquid added to the mouth area. In various embodiments, the resulting disintegration time may be between about 0 and about 300 seconds, between about 0 to about 60 seconds, less than about 30 seconds or less than about 15 seconds in the oral cavity.

A systemically acting pharmaceutical ingredient is a compound that may be taken by mouth and acts systemically to treat various conditions. Systemic agents may take up to about 30 minutes or more after consumption to provide an effect on a patient.

“Sensory cue” is defined herein as perceptible sensations such as prickling, burning, cooling, numbing, heating, vapor action, to a point wherein the patient does not find such sensations objectionable. A sensory cue agent is desirably present in an a sensory effective amount which is an amount that imparts an immediately perceivable effect. Sensory agents may provide an effect on the thermoreceptors in the mucosal cavities, such as those in a buccal/oral cavity and a nasal passage. Sensory agents may also provide an effect on the pain receptors in the mouth or throat area. Volatile and nonvolatile sensory agents may be used depending on the desired effect. A volatile sensory cue agent is defined as any compound having the property of being volatile and being able to stimulate thermoreceptors of the nervous system to produce cold or heat sensations.

Useful sensory agents include, but are not limited to, menthol, including menthol oil and menthol crystals, mannitol, eucalyptus oil (or eucalyptol), thymol, camphor, spearmint, cinnamon, mint, ginger, wintergreen (methyl salicylate), peppermint, carboxamides and combinations thereof. Useful carboxamides include 3-1-menthoxy propane-1,2-diol, N-substituted-p-menthane-3-carboxamides and acyclic carboxamides and combinations thereof and are disclosed in U.S. Pat. Nos. 4,136,163, 4,230,688 and 4,459,425, which are incorporated herein by reference in their entirety. Preferred volatile aromatic include N-ethyl-p-menthane-3-carboxamide which is commercially available as WS-3 and N,2,3-trimethyl-2-isopropylbutanamide which is commercially available as WS-23 from Wilkinson Sword Limited.

Sensory cue agents may be present at a level of from about 0.001% to about 15%, or from about 0.001% to about 5%, more preferably from about 0.001% to about 0.5% by weight of the pharmaceutical compositions. Useful amounts of a sensory agent include between about 2 mg to about 15 mg, between about 4 mg to about 10 mg, about 5 mg or about 10 mg per dosage form. However, the exact amount of sensory agent employed is a matter of preference subject to such factors as the degree of sensory effect desired. Thus, the amount of a sensory agent can be varied in order to obtain the result desired in the final product and such variations are within the capabilities of those skilled in the art without the need for undue experimentation.

A useful sensory agent is menthol including menthol that is obtained from sources of menthol oil and menthol crystals. Menthol is a volatile sensory agent that may produce an immediate perceivable “sensory cue” effect in the mouth, nasal and sinus passages. Menthol may exert a nasal decongestion effect, cough suppression, oral anesthetic and/or antitussive action. Menthol may produce a physiological cooling effect on the mucous membranes of the body, particularly those of the mouth, nose, throat and gastrointestinal tract. Menthol is a Generally Recognized as Safe (GRAS) and Effective (GRAS/E) ingredient and is denoted “Category I” as a cough suppressant or antitussive. A number of clinical studies provide support for use of menthol as a nasal decongestant. See U.S. Food & Drug Administration (FDA) Sep. 9, 1976. Establishment of a monograph for OTC cold, cough, allergy, bronchodilator and antiasthmatic products. Fed. Reg. 41:38312-38424; U.S. FDA. Aug. 12, 1988. Cold, cough, allergy, bronchodilator and antiasthmatic products for over-the-counter human use. Tentative Final Monograph for Combination Drug Products. Fed. Reg. 53:30522-30564; C L Blanchard, et al., Evaluation of nasal decongestant drugs. 1964 The Eye, Ear, Nose & Throat Monthly 43:76-82; V Schulz, R Hansel, V E Tyler. Rational Phytotherapy, 3^{rd} ed. Berlin, Springer Verlag, 1998, 146-7, each of which is incorporated herein by reference in its entirety. Useful amounts of menthol include between about 2 mg to about 15 mg, between about 4 mg to about 10 mg, about 5 mg or about 10 mg per FDDF.

In one embodiment, the at least one sensory cue agent includes a menthol and eucalyptus oil combination. Eucalyptus oil imparts a nasal decongestant activity.

Various embodiments of the present invention provide compositions with at least one systemic API, however, other embodiments include compositions with at least two systemic API's and even with at least three systemic API's.

Useful systemic API's, include, but are not limited to, therapeutically effective amounts of antihistamines, decongestants, bronchodilators, expectorants, antitussives, analgesics, demulcents, anesthetics, antiviral agents, antiseptics, antibiotics, immune enhancing ingredients, vitamins and combinations thereof.

Useful systemic API's include, but are not limited to:
(a) antimicrobial agents such as triclosan, cetylpyridium chloride, domiphen bromide, quaternary ammonium salts, zinc compounds, sanguinarine, fluorides, alexidine, octonidine, EDTA, and the like;
(b) non-steroidal anti-inflammatory and pain reducing agents such as aspirin, acetaminophen, ibuprofen, ketoprofen, diflunisal, fenoprofen calcium, flurbiprofen sodium, naproxen, tolmetin sodium, indomethacin, celecoxib, valdecoxib, parecoxib, rofecoxib and the like;
(c) antitussives such as benzonatate, caramiphen edisylate, menthol, dextromethorphan hydrobromide, clofedanol, carbetapentane, noscapine, codeine, chlophedianol hydrochloride and the like;
(d) antihistamines such as brompheniramine maleate, chlorpheniramine maleate, carbinoxamine maleate, clemastine fumarate, dexchlorpheniramine maleate, diphenylhydramine hydrochloride, azatadine maleate, diphenhydramine citrate, diphenhydramine hydrochloride, diphenylpyraline hydrochloride, doxylamine succinate, promethazine hydrochloride, pyrilamine maleate, tripelennamine citrate, triprolidine hydrochloride, acrivastine, loratadine, desloratadine, brompheniramine, dexbropheniramine, fexofenadine, cetirizine, levo-cetirizine, dextro-cetirizine, chlorcycline, alimemazine, pyrilamine, montelukast sodium and the like;
(e) expectorants such as guaifenesin, ipecac, potassium iodide, terpin hydrate, bromhexine, carbocysteine, potassium guaicol sulfonate and the like;
(f) analgesic-antipyretics such salicylates, phenylbutazone, indomethacin, phenacetin and the like;
(g) antimigraine drugs such as sumitriptan succinate, zolmitriptan, valproic acid eletriptan hydrobromide and the like;
(h) antiviral agents including zinc;
(g) demulcents including pectin, glycerin or honey;
(h) vitamins including vitamin C, Vitamin E;
(i) bronchodilators including methylxanthines, epinephrine, racepinephrine;
(j) antiseptics including cetylpyridinium chloride;
(k) antibiotics including cephalexin and amoxicillin;
(l) anesthetics including benzocaine, cetacaine, lidocaine;
(m) immune enhancing ingredients including herbs (e.g. echinacea, ginseng, astragalus, schisandra, and andrographis), and propolis;
(n) anticholinergics including ipratropium and combinations thereof.

The amount of the systemic API's in the formulation may be adjusted to deliver a predetermined dose of the active agent over a predetermined period of time, which may typically vary from 4 to 24 hours. Examples of doses containing specific APIs are set forth in Table 1.

TABLE 1

| Pharmaceutically Active Agent | Dose | |
|---|---|---|
| Chlorpheniramine Maleate | 4-12 | mg |
| Brompheniramine Maleate | 4 | mg |
| Dexchlorpheniramine | 2 | mg |
| Dexbropheniramine | 2 | mg |
| Triprolidine Hydrochloride | 2.5 | mg |
| Cetirizine | 5-10 | mg |
| Acrivastine | 8 | mg |
| Azatadine Maleate | 1 | mg |
| Loratadine | 5-10 | mg |
| Dextromethorphan Hydrobromide | 10-30 | mg |
| Ketoprofen | 12.5-25 | mg |
| Sumatriptan Succinate | 35-70 | mg |
| Zolmitriptan | 2.5 | mg |
| Nicotine | 1-15 | mg |
| Diphenhydramine Hydrochloride | 12.5-25 | mg |
| Atorvastatin | 5-80 | mg |
| Valdecoxib | 5-20 | mg |
| Celecoxib | 5-20 | mg |
| Rofecoxib | 5-25 | mg |
| Ziprasidone | 20-80 | mg |
| Eletriptan | 10-40 | mg |

Except as otherwise noted, the amount of a systemic API is designated as % by weight per dosage form. Generally, the amount of the systemic API used may be from about 0.01% to about 80% by weight, or from about 0.1% to about 40% by weight, or from about 1% to about 30% by weight, or from about 1% to about 10% by weight.

In certain embodiments, particularly useful systemic API's include pseudoephedrine, phenylephrine, ephedrine, phenylpropanolamine, dextromethorphan, diphenhydramine, isomers thereof, prodrugs thereof, pharmaceutically acceptable salts thereof or a pharmaceutically acceptable salts of said prodrug thereof and combinations thereof. Pseudoephedrine, phenylephrine, dextromethorphan and diphenhydramine may be administered in the form of its hydrochloride salt but can be present in the form of its free base or any pharmaceutically acceptable salt, e.g., citrate, maleate, hydrobromide, tannate. Useful amounts of pseudoephedrine include from about 15 mg to about 360 mg; from about 15 mg to about 60 mg; from about 30 to about 60 mg; or about 30 mg; or about 60 mg. Useful amounts of phenylephrine include from about 2.5 mg to about 50 mg from about 5 to about 25 mg; from 5 about to about 10 mg; or about 10 mg. Useful amounts of dextromethorphan include from about 2.5 mg to about 60 mg; from about 5 to about 20 mg; from about 7.5 to about 15 mg or about 7.5 mg; or about 15 mg. Useful amounts of diphenhydramine include from about 1 mg to about 100 mg; from about 5 to about 50 mg; from about 12.5 to about 50 mg or about 12.5 mg; or about 25 mg.

Various embodiments of the present invention can be administered for the reduction, treatment, management or mitigation of ailments such as upper respiratory indications, including but not limited to, nasal congestion and cough, cold, cold-like symptoms, symptoms related to upper respiratory infections, influenza, asthma, allergies or allergic reactions, allergic and perennial rhinitis, sinusitis, Eustachian tube congestion and combinations thereof.

An “effective” amount or a “therapeutically effective amount” of an active ingredient refers to a non-toxic but sufficient amount of the agent to provide the desired effect. The amount of active agent that is “effective” will vary from subject to subject, depending on the age and general condition of the individual, the particular active agent or agents, and the like. Thus, it is not always possible to specify an exact “effective amount.” However, an appropriate “effective” amount in any individual case can be determined by one of ordinary skill in the art using routine experimentation.

“Pharmacologically active” (or simply “active”), refers to a compound that has pharmacological activity and a “pharmacologically active” derivative of an active agent, refers to a derivative having the same type of pharmacological activity as the parent compound and approximately equal in degree. When the term “pharmaceutically acceptable” is used to refer to a derivative (e.g., a salt) of an active agent, it is to be understood that the compound is pharmacologically active as well. When the term “pharmaceutically acceptable” is used to refer to an excipient, it implies that the excipient has met the required standards of toxicological and manufacturing testing or that it is on the Inactive Ingredient Guide prepared by the Food and Drug Administration.

By “pharmaceutically acceptable” such as in the recitation of a “pharmaceutically acceptable excipient,” or a “pharmaceutically acceptable additive,” is meant a material that is not biologically or otherwise undesirable, i.e., the material can be incorporated into a pharmaceutical composition administered to a patient without causing any undesirable biological effects or interacting in a deleterious manner with any of the other components of the composition in which it is contained.

In various embodiments of the present invention, the dosage forms may be administered orally. Oral administration may involve swallowing, so that the the systemic API(s) enters the gastrointestinal tract, and/or buccal, lingual, or sublingual administration by which the API enters the blood stream directly from the mouth.

Useful dosage forms of the pharmaceutical compositions include orally fast disintegrating systems including, but not limited to solid, semi-solid and liquid systems including fast disintegrating or fast dissolving tablets, soft or hard capsules, gels, fast dispersing dosage forms, caplets, films, wafers, ovules, granules, buccal/mucoadhesive patches, powders, freeze dried (lyophilized) wafers, chewable tablets which disintegrate with saliva in the buccal/mouth cavity and combinations thereof. Useful films include, but are not limited to, single layer stand alone films and dry multiple layer stand alone films.

Liquid formulations include suspensions, solutions, syrups and elixirs may be employed as fillers in soft or hard capsules including those made, for example, from gelatin or hydroxypropylmethylcellulose and typically comprise a carrier, for example, water, ethanol, polyethylene glycol, propylene glycol, methylcellulose, or a suitable oil, and one or more emulsifying agents and/or suspending agents.

In one embodiment, a fast disintegrating dosage form is contemplated where a dry mixture of the components of the invention gives rise, upon direct compression, to fast disintegrating tablets. In several embodiments, it is useful to use fast-dissolving, fast-disintegrating dosage forms such as those described in U.S. Pat. No. 5,576,014 and Expert Opinion in Therapeutic Patents, 11 (6), 981-986, by Liang and Chen (2001), which are all incorporated herein in their entirety.

Useful inactive ingredients, include but are limited to, binding agents, filling agents, lubricating agents, suspending agents, sweeteners, flavorings and flavor enhancer agents, taste-masking agents, preservatives, buffers, wetting agents, anti-oxidants, colorants or coloring agents, pharmaceutically acceptable carriers, disintegrants, salivary stimulating agents, cooling agents, co-solvents (including oils), pH adjusting agents, effervescent agents, emollients, bulking agents, anti-foaming agents, surfactants, soluble organic salts, permeabilizing agents, glidants and other excipients and combinations thereof. Desirably, the agents are chemically and physically compatible with the API.

Useful pH adjusting agents include fumaric acid, citric acid, sodium acetate. Useful surfactants include sorbitan esters, docusate sodium, sodium lauryl sulfate, cetriride. Useful soluble organic salts include sodium carbonate, sodium bicarbonate, sodium chloride.

Examples of useful binding agents include, but are not limited to, polyethylene glycols, soluble hydroxyalkylcelluloses, polyvinylpyrrolidone, gelatins, natural gums, various celluloses and cross-linked polyvinylpyrrolidone, microcrystalline cellulose, such as Avicel® PH101 and Avicel® PH102.

Examples of useful substantially water soluble carriers or filling agents include, but are not limited to, various starches, celluloses, carbohydrates compression sugars or soluble fillers. More particularly, useful fillers include but are not limited to lactose, lactose monohydrate, lactose anhydrous, sucrose, amylose, dextrose, mannitol, inositol, maltose, maltitol, sorbitol, glucose, xylitol, erythritol, fructose, maltodextrins; microcrystalline cellulose, calcium carboxy methyl cellulose; pregelatinized starch, modified starches, potato starch, maize starch; clays, including kaolin and polyethylene glycols (PEG) including PEG 4000; or combinations thereof. Useful amount of fillers include the range of about 1 to about 99 weight percent, or about 25 to about 95 weight percent or about 40 weight percent to about 95 weight percent of the compositions of this invention. Microcrystalline cellulose may also be used for its properties as a filler and plasticizing agent and, therefore, also can be regarded as a substantially water insoluble excipient.

Compositions of the present invention may include a sweetener. Useful sweeteners include, but are not limited to, sugars such as sucrose, glucose (corn syrup), dextrose, invert sugar, fructose, and mixtures thereof; acid saccharin and its various salts such as the sodium or calcium salt; cyclamic acid and its various salts such as the sodium salt; the dipeptide sweeteners such as aspartame and alitame; natural sweeteners such as dihydrochalcone compounds; glycyrrhizin; Stevia rebaudiana (Stevioside); sugar alcohols such as sorbitol, sorbitol syrup, mannitol, xylitol and the like, synthetic sweeteners such as acesulfame-K and sodium and calcium salts thereof and other synthetic sweeteners, hydrogenated starch hydrolysate (lycasin); protein based sweetening agents such as talin (thaumaoccous danielli) and/or any other pharmacologically acceptable sweetener known by the state of the art, and mixtures thereof.

Suitable sugar alcohols useful as sweeteners include, but are not limited to, sorbitol, xylitol, mannitol, galactitol, maltitol, isomalt (PALATINIT™) and mixtures thereof. The exact amount of sugar alcohol employed is a matter of preference subject to such factors as the degree of cooling effect desired. Thus, the amount of sugar alcohol may be varied in order to obtain the result desired in the final product and such variations are within the capabilities of those skilled in the art without the need for undue experimentation.

In another embodiment, the formulations according of the invention are free of sugar. A sugar-free formulation has the advantage that it can be administered easily to consumers with blood sugar disorders or to diabetics in need of such preparations. Such sweeteners include, but are not limited to, sucralose, acesulfame potassium, and aspartame which share properties such as absence of bitter and metallic aftertastes.

In another embodiment, a composition may include acesulfame K, aspartame, sucralose and combinations thereof. Acesulfame K is a commercial product of Nutrinova Nutrition Specialties & Food Ingredient GmbH. Useful amounts of sucralose in a dosage form is between about 0.002% to about 10% by total weight of the FDDF. However, this amount can vary greatly depending upon the nature of the composition being sweetened. In one preferred embodiment, the sweetener is a mixture of sucralose with acesulfame K.

The tablets may be uncoated, however, they can, if desired, be coated with any suitable coating agent known in the art. Suitable coating agents are those used for immediate release purposes and will disintegrate in saliva. Such coatings include, but are not limited to, hydroxypropyl methylcellulose, or methyl cellulose, or OPADRY™ and the like and combinations thereof.

Optionally, one or more flavors such as those described in U.S. Pat. No. 6,596,298 which is incorporated herein. Any amount of flavor can be used and will depend on characteristics of the active pharmaceutical ingredient(s); preferred concentration of flavoring is between about 0.01% to about 10% w/w of a composition.

A tablet disintegrant may be added to the direct compression process for its wicking (i.e., the ability of particles to draw water into the porous network of a tablet) and swelling ability. Some disintegrants also serve as excellent binders and are able to substantially improve the mechanical strength of the formulation. Suitable disintegrants are carboxymethyl cellulose sodium, carboxymethyl cellulose calcium, crospovidone, sodium starch glycolate, corn starch, insoluble cationic-exchange resins such as polyacrylin, microcrystalline cellulose, croscarmellose. Disintegrants can be added at a concentration ranging from about 0.5% to about 50%. Croscarmellose sodium (cross-linked carboxymethyl cellulose) may be present at a concentration of about 2% to about 10%.

An effective amount of any generally accepted pharmaceutical tableting lubricant can be added to compress the tablets. An amount within the range from about 0.25% to about 6%, or 0.5% to about 3% by weight can be added. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

One or more glidant materials which improve the flow of the powder blend and minimize the dosage form weight variation can be used. Useful glidants include but are not limited to silicone dioxide, talc and combinations thereof.

Certain embodiments of the invention can further provide a taste-masked oral pharmaceutical composition including coating or encapsulating the systemically active therapeutic agent with a suitable coating material. Examples of suitable coating materials for taste-masking include polymers such as hydroxypropylmethylcellulose, ethylcellulose, methacrylates, methacrylate co-polymers such as Eudragit® (Butylmethacrylat-(2-Dimethylaminoethyl)methacrylat-Methylmethacrylat-Copolymer (1:2:1)”), KOLLICOAT®, and polyvinylpyrrolidone. The pharmaceutical composition can include other functional components presented for the purpose of modifying the physical, chemical or taste properties of the systemically active therapeutic agent. For example, the systemically active therapeutic agent can be in the form of microencapsulation, ion-exchange resin complex, such as a sulfonated polymers, electro-chemical melt, supercritical fluids, magnesium trisilicate, coacervation, or cyclodextrin (cyclic-linked oligosaccharides) complexes. Useful sulphonated polymers include polystyrene cross-linked with 8% of divinylbenzene such as Amberlite® IRP-69 and IRP-64 (obtained by Rohm and Haas), Dow XYS-40010.00®, Dow XYS40013.00® (obtained from the Dow Chemical Company).

The dose, pKa and solubility of the drug molecule influences formulation and taste masking methods. It is understood that any method in the art for masking the taste of pharmaceuticals to facilitate their oral administration can be used. For example, taste masking can also be achieved by simple wet granulation or roller compaction with other excipients to minimize presented surface area of the drug. Spray drying can also be used to taste mask the systemically active therapeutic agent.

It is further contemplated that the pharmaceutically active ingredients can be added in the form of an encapsulate. Encapsulation can be achieved using conventional procedures and can be performed using water-insoluble as well as water-soluble agents. Alternatively, it is possible to encapsulate a release controlling substance, together with the systemically active therapeutic agent, within an encapsulating shell to provide for controlled release of the taste-masked oral pharmaceutical composition.

An embodiment of the present invention provides for a process for preparing a tablet formulation. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. Freeze or spray drying may also be used. The formulation of tablets is discussed in Pharmaceutical Dosage Forms: Tablets, Vol. 1, by H. Lieberman and L. Lachman (Marcel Dekker, New York, 1980).

Direct compression is a relatively quick process where the powdered materials are compressed directly without changing the physical and chemical properties of the drug. Direct compression excipients are chosen such that they have good flow and compressible characteristics and prevent segregation of powders in the hopper and thereby help in direct compression. For example, tablets may be obtained by blending together the API(s) and sensory cue agents, and optional inactive ingredients, and optionally other therapeutically active ingredients and excipients to form a homogeneous mixture; blending together; and directly compressing the mixture.

In another embodiment, the dosage form composition is a standalone film prepared by any suitable method for producing fast dissolving film such as those described in U.S. Pat. No. 6,596,298 issued to Leung et al, which is incorporated herein. Consumable oral films for human or veterinary use are typically pliable water-soluble or water-swellable thin film dosage forms which may be rapidly dissolving or mucoadhesive and typically include an API, a film-forming polymer, a binder, a solvent, a humectant, a plasticiser, a stabiliser or emulsifier, a viscosity-modifying agent and a solvent. Some components of the formulation may perform more than one function. Films may be manufactured by conventional processes such as those disclosed in U.S. Pat. Nos. 3,784,390; 4,927,636; 6,177,096, each of which is incorporated herein. The film-forming polymer may be selected from natural polysaccharides, proteins, or synthetic hydrocolloids and is typically present in the range 0.01 to 99 weight %, more typically in the range 30 to 80 weight %. Useful water soluble film forming polymers are described in U.S. Pat. No. 6,596,298 to Leung et al. and include, but are not limited to, polyvinyl alcohol, pullulan, hydroxypropylmethylcellulose, hydroxyethylcellulose, hydroxypropylcellulose, polyvinylpyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. A particularly useful water soluble polymer is pullulan. The dry film can be cut to suitable size and shape for unit dose pouching.

Solid formulations for oral administration may be formulated to be immediate and/or modified controlled release. Controlled release formulations include modified release formulations include delayed-, sustained-, pulsed-, controlled-, targeted and programmed release.

Suitable modified release formulations for the purposes of the invention are described in U.S. Pat. No. 6,106,864, which is incorporated herein. Details of other suitable release technologies such as high energy dispersions and osmotic and coated particles are to be found in Pharmaceutical Technology On-line, 25(2), 1-14, by Verma et al (2001), which is incorporated herein. The use of chewing gum to achieve controlled release is described in WO 00/35298, which is incorporated herein.

Another embodiment of the present invention provides a kit having two or more separate compositions having a systemic API and sensory cue agent and a means for separately retaining said compositions, such as a container, divided bottle, or divided foil packet. An example of such a kit is the familiar blister pack used for the packaging of tablets, capsules and the like. Other embodiments contemplate articles of manufacture including various packaging configurations, ranging from unit dose blister packs to multiple dose packages such as bottles. To assist compliance, the kit may have directions for administration and may be provided with a so-called memory aid. Another embodiment contemplates a method of dispensing a composition from a blister pack by forcing the drug product through a foil back on a blister pack.

While certain preferred and alternative embodiments of the invention have been set forth for purposes of disclosing the invention, modification to the disclosed embodiments can occur to those who are skilled in the art and are contemplated by the present invention and are within the scope of the present invention.

The following examples serve to provide further appreciation of the invention but are not meant in any way to restrict the effective scope of the invention.

### EXAMPLES

Pharmaceutical compositions with the formulations set forth in Table 2 are manufactured by blending a dry mixture of the active systemic pharmaceutical ingredients with the other pharmaceutically acceptable excipients, and then adding the sensory cue agent(s) to the homogeneous mixture. The tablets are then prepared by direct compaction of the pharmaceutical compositions. The resulting tablets disintegrate in the buccal cavity within 60 seconds and provide an immediate sensory cue to the oral and nasal cavities. The ingredients of the examples are in milligrams unless otherwise noted.

TABLE 2

| Ingredient/Example (mg) | 1 | 2 | 3 | 4 | 5 | 6 | 7 |
|---|---|---|---|---|---|---|---|
| taste masked diphenhydramine citrate (60% active) | 31.67 | | | | | | |
| phenylephrine hydrochloride | | 5.00 | | | 10.00 | | |
| pseudoephedrine hydrochloride encapsulated (50%) | | | 60.00 | | | | |
| dextromethorphan hydrobromide adsorbate (20%) | | | | 100.00 | | | |
| chlorpheniramine maleate | | | | | 4.00 | | |
| pseudoephedrine hydrochloride encapsulated (63% active) | | | | | | 47.62 | 47.62 |
| dextromethorphan hydrobromide adsorbate (48%) | | | | | | 31.25 | |
| ibuprofen encapsulate (82% active) | | | | | | | 243.90 |
| sucralose | 8.00 | 2.00 | | 3.15 | 3.00 | | 4.00 |
| saccharine | | | | | | 3.75 | |
| aspartame | | | | | 2.00 | 3.75 | 2.00 |
| acesulfame potassium | | 2.00 | 4.00 | 2.75 | | | |
| cherry flavor | | 5.00 | | | | | |
| strawberry flavor | 8.00 | | | | | | |
| spearmint flavor | | | | 8.25 | | | |
| peppermint flavor | | | | | 7.40 | | |
| anise flavor | | | | | | 6.00 | |
| 3-1 methoxy propane-1,2-diol | | | | 1.35 | | | |
| cinnamic aldehyde | | | | | | | 6.00 |
| Menthol | 5.00 | 6.00 | 4.75 | | 12.00 | 6.25 | 10.00 |
| eucalyptus oil adsorbate (25% active) | | | 7.50 | | | 6.00 | 5.00 |
| citric acid | 4.00 | 4.00 | 5.00 | 3.80 | 4.60 | 3.00 | 3.00 |
| magnesium stearate | 2.00 | 1.00 | 1.00 | 1.00 | 1.50 | 1.00 | 2.00 |
| lactitol, monohydrate | 250.00 | 175.00 | 269.00 | 173.00 | 315.00 | 356.00 | 405.00 |
| xylitol | | | | | | | 68.48 |
| mannitol | 84.33 | 44.50 | 43.00 | 44.00 | 37.00 | 29.63 | |
| stearic acid | 6.00 | 2.00 | 5.00 | 2.00 | 3.00 | 2.00 | 8.00 |
| monoammonium glycyrrhizinate | | | | | | 1.75 | 3.00 |
| FD&C Red #40 Al Lake | 1.00 | 0.50 | | | | | |
| FD&C Blue #1 Al Lake | | | 0.75 | | 0.50 | | |
| FD&C Yellow #10 Al Lake | | | | | | 1.25 | 1.25 |
| FD&C Yellow #6 Al Lake | | | | | | 0.75 | 0.75 |
| FD&C Green #3 Al Lake | | | | 0.70 | | | |

1. A pharmaceutical composition comprising:
(a) at least one systemically active pharmaceutical ingredient in a therapeutically effective amount; and
(b) at least one sensory cue agent in a sensory cue effective amount;
wherein said pharmaceutical composition is designed to disintegrate in the buccal cavity in less than about 60 seconds.

2. The composition of claim 1 wherein the sensory cue agent is selected from the group consisting of menthol oil, menthol crystals, mannitol, eucalyptus oil, eucalyptol, thymol, camphor, spearmint, cinnamon, mint, ginger, wintergreen (methyl salicylate), peppermint, carboxamides, acyclic carboxamides, 3-1-menthoxy propane-1,2-diol, N-substituted-p-menthane-3-carboxamides, N-ethyl-p-menthane-3-carboxamide, N,2,3-trimethyl-2-isopropylbutanamide and combinations thereof.

3. The composition of claim 1 wherein said sensory cue agent is present in an amount from about 2 mg to about 15 mg.

4. The composition of claim 1 wherein said sensory cue agent is present in an amount from about 0.001% to about 15% by weight of the pharmaceutical composition.

5. The composition of claim 1 wherein said composition disintegrates in said mouth in less than 30 seconds.

6. The composition of claim 1 wherein said sensory cue agent is menthol.

7. The composition of claim 1 wherein said sensory cue agent comprises menthol and eucalyptus.

8. The composition of claim 1 wherein said active pharmaceutical agent is selected from the group consisting of phenylephrine, pseudoephedrine, phenylpropanolamine, ephedrine, dextromethorphan, diphenhydramine, a prodrug thereof, an isomer thereof, a stereoisomer thereof, a pharmaceutically acceptable salt thereof, or a pharmaceutically acceptable salt of said prodrug thereof and combinations thereof.

9. The composition of claim 8 wherein said pseudoephedrine is present in an amount of from about 15 mg to about 360 mg.

10. The composition of claim 8 wherein said phenylephrine is present in an amount from about 2.5 mg to about 50 mg.

11. The composition of claim 8 wherein said dextromethorphan is present in an amount from about 2.5 mg to about 50 mg.

12. The composition of claim 8 wherein said diphenhydramine is present in an amount from about 2.5 mg to about 50 mg.

13. The composition of claim 1, wherein said active pharmaceutical ingredient is selected from the group consisting of phenylephrine, pseudoephedrine, phenylpropanolamine, ephedrine, dextromethorphan, diphenhydramine, a prodrug thereof, an isomer thereof, a stereoisomer thereof, a pharmaceutically acceptable salt thereof, or a pharmaceutically acceptable salt of said prodrug thereof and combinations thereof and wherein said sensory cue agent is selected from the group consisting of menthol oil, menthol crystals, mannitol, eucalyptus oil, eucalyptol, thymol, camphor, spearmint, cinnamon, mint, ginger, wintergreen (methyl salicylate), peppermint, carboxamides, acyclic carboxamides, 3-1-menthoxy propane-1,2-diol, N-substituted-p-menthane-3-carboxamides, N-ethyl-p-menthane-3-carboxamide, N,2,3-trimethyl-2-isopropylbutanamide and combinations thereof.

14. The composition of claim 1 wherein said composition is in a dosage form selected from the group consisting of a tablets, soft or hard capsules, gels, caplets, wafers, ovules, granules, buccal/mucoadhesive patches, powders, freeze dried (lyophilized) wafers, chewable tablets, single layer stand alone films, dry multiple layer stand alone films and combinations thereof.

15. The composition according to claim 1 wherein the systemically active pharmaceutical ingredient is selected from the group consisting of antihistamines, decongestants, antitussives, anesthetics, expectorants, demulcents, immune stimulators, vitamins, antibiotics, antiseptics, analgesics, bronchodilators, and combinations thereof.

16. The composition according to claim 1 wherein the systemically active pharmaceutical ingredient is selected form the group consisting of pseudoephedrine, phenyleprine, dextromethorphan, benzocaine, cetacaine, lidocaine, cetylpyridinium chloride, diphenhydramine, chlorpheniramine, acetaminophen, ibuprofen, aspirin, naproxen, celecoxib, valdecoxib, rofecoxib, brompheniramine, dexbrompheniramine, tripolidine, dexchlorpheniramine, chlorcyclizine, triprolidine, doxylamine, carbinoxamine, azatadine; tripelennamine, alimemazine, bromodiphenhydramine, phenindamine, pyrilamine, loratadine, desloratadine, fexofenadine, cetirizine, acrivastine, levocetirizine, mizolastine, codeine, carbetapentane, caramiphen, noscapine, clofedanol, guaifenesin, bromhexine, carbocysteine, terpin hydrate, potassium guaicolsulfonate, zinc, glycerin, honey, vitamin C, vitamin E, methylxanthines, epinephrine, racepinephrine, cetapyridium, cephalexin, amoxicillin, phenol and combinations thereof.

17. A pharmaceutical composition comprising:
(a) at least one systemically active pharmaceutical ingredient in a therapeutically effective amount; wherein said active pharmaceutical ingredient is selected from the group consisting of phenylephrine, pseudoephedrine, dextromethorphan, diphenhydramine and combinations thereof and
(b) at least one sensory cue agent in a sensory cue effective amount; wherein said sensory cue agent is selected from the group consisting of menthol oil, menthol crystals, mannitol, eucalyptus oil, eucalyptol, thymol, camphor, spearmint, cinnamon, mint, ginger, wintergreen (methyl salicylate), peppermint, carboxamides, acyclic carboxamides, 3-1-menthoxy propane-1,2-diol, N-substituted-p-menthane-3-carboxamides, N-ethyl-p-menthane-3-carboxamide, N,2,3-trimethyl-2-isopropylbutanamide and combinations thereof;
wherein said pharmaceutical composition is a tablet designed to disintegrate in the buccal cavity in less than about 60 seconds.

18. The composition of claim 17, wherein said sensory cue agent is selected from the group consisting of menthol and eucalyptol and combinations thereof.

19. A method for treating upper respiratory ailments in humans and animals, comprising administering a composition according to claim 17 to a patient in need of such treatment.

20. A packaged kit for a patient comprising:
a housing of a plurality of oral dosage forms comprising a pharmaceutical composition comprising:
(a) at least one systemically active pharmaceutical ingredient in a therapeutically effective amount; and
(b) at least one sensory cue agent in a sensory cue effective amount;
wherein said pharmaceutical composition is designed to disintegrate in the buccal cavity in less than about 60 seconds, and;
instructions for carrying out drug administration therewith.
